(19) United States
(12) Patent Application Publication
(10) Pub. No.: US 2004/0063729 A1
(43) Pub. Date: Apr. 1, 2004

(54) Triazolopyrimidines

(76) Inventors: Yoshinori Kitagawa, Tochigi (JP); Haruko Sawada, Ibaraki (JP); Yutaka Kuchii, Tochigi (JP)

Correspondence Address: BAYER CROPSCIENCE LP, Patent Department, 100 BAYER ROAD, PITTSBURGH, PA, 15205-9741, US

(21) Appl. No.: 10/450,996

(22) PCT Filed: Dec. 12, 2001
(86) PCT No.: PCT/IB01/02441

(30) Foreign Application Priority Data
Dec. 22, 2000 (JP) 2000390475
Jun. 11, 2001 (JP) 2001175427

(51) Int. Cl. A61K 31/519; C07D 487/02

(52) U.S. Cl. 514/259.31; 544/255

(57) ABSTRACT

The invention relates to novel triazolopyrimidines of the formula

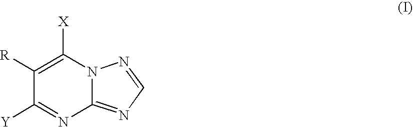

wherein

X represents halogen,

Y represents a hydrogen atom or halogen, and

R has the meanings given in the disclosure,

to a process for the preparation of the new compounds, and to their use as microbicides.

The present invention relates to novel pyrazolopyrimidines, to a process for their preparation and to their use as microbicides.

It has already been known that certain triazolopyrimidines can be employed for the control of fungi (cf. JP-A 507 505-1996 and JP-A 124 651-1997). The fungicidal activity of such known compounds, however, is not always satisfactory.

Further, it has already been described that certain triazolopyrimidines can be used as angiotensin II receptor antagonists (cf. JP-A 504 178-1995), as intermediates for the preparation of drugs and herbicides (cf. DD-A 70 311) or as agents for the dilatation of the heart coronal artery (cf. GB-A 1 148 629, DD-A 55 956, DD-A 61 289 and DD-A 99 974).

There have now been found novel triazolopyrimidines of the formula

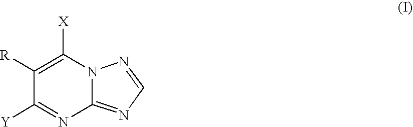

wherein

X represents halogen,

Y represents a hydrogen atom or halogen, and

R represents phenyl-C₁₋₄ alkyl optionally substituted by one or more radicals selected from halogen, alkyl alkenyl, alkylene, dialkylamino, alkoxy, alkyl-carbonyl having 1 to 4 carbon atoms in the alkyl group, alkoxycarbonyl having 1 to 4 carbon atoms in the alkoxy group, alkylthio, haloalkyl, haloalkoxy, haloalkylthio, phenyl, benzyl, phenoxy, cyano, nitro and thiadiazolyl,

or

R represents diphenylmethyl optionally substituted by one or more radicals selected from halogen, alkyl, alkenyl, alkylene, dialkylamino, alkoxy, alkyl-carbonyl having 1 to 4 carbon atoms in the allyl group, alkoxycarbonyl having 1 to 4 carbon atoms in the alkoxy group, alkylthio, haloalkyl, haloalkoxy, haloalkylthio, phenyl, benzyl, phenoxy, cyano, nitro and thiadiazolyl,

or

R represents naphthylmethyl optionally substituted by one or more radicals selected from halogen, alkyl, alkenyl, alkylene, dialkylamino, alkoxy, alkyl-carbonyl having 1 to 4 carbon atoms in the alkyl group, alkoxycarbonyl having 1 to 4 carbon atoms in the alkoxy group, alkylthio, haloalkyl, haloalkoxy, haloalkylthio, phenyl, benzyl, phenoxy, cyano, nitro and thiadiazolyl, or

R represents anthranyl-methyl.

Further, it has been found that the triazolopyrimidines of the formula (I) can be prepared by

a) reacting in a first step compounds of the formula

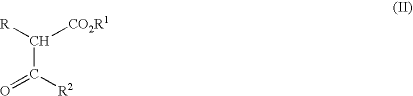

wherein

R has the above-mentioned meanings,

R¹ represents C₁₋₄ alkyl, and

R² represents a hydrogen atom or C₁₋₄ alkoxy,

with 3-amino-1,2,4-triazole of the formula

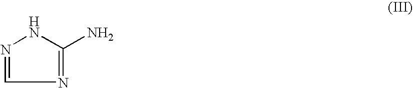

in the presence of an inert diluent and, if appropriate, in the presence of an acid-binding agent or of an acid catalyst,

and

b) reacting in a second step the triazolopyrimidines thus obtained having the formula

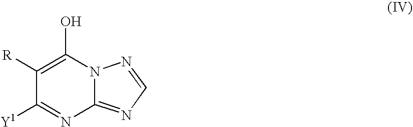

wherein

R has the above-mentioned meanings and

Y¹ is a hydrogen atom or hydroxy,

with halogenating agents in the presence of an inert diluent.

Finally, it has been found that the triazolopyrimidines of the formula (I) are out-standingly active as microbicides in agriculture and horticulture as well as for the preservation of materials.

Surprisingly, the triazolopyrimidines of the formula (I) according to the invention have a much better microbicidal activity than the already known compounds, which are structurally most similar and have the same type of action.

In the present specification:

“Halogen” represents fluoro, chloro, bromo or iodo and preferably represents, fluoro, chloro or bromo.

“Alkyl” can be straight-chain or branched-chain and there may be mentioned, for example, methyl, ethyl, n- or iso-propyl, n-, iso-, sec- or tert-butyl and so on.

“Alkenyl” can be straight-chain or branched-chain and there may be mentioned, for example, vinyl, allyl, isopropenyl, 1-propenyl, 1-butenyl, 2butenyl, 3-butenyl, 1-methyl-1-propenyl, 2-methyl-1-propenyl and so on.

“Alkylene”includes, double-bonded groups, such as trimethylene, tetramethylene and so on.

“Alkoxy” can be straight-chain or branched-chain and there may be mentioned, for example, methoxy, ethoxy, n- or iso-propoxy, n-, iso-, sec- or tert-butoxy and so on.

“Alkylthio” can be straight-chain or branched-chain and there may be mentioned, for example, methylthio, ethylthio, n-or iso-propylthio, n-, iso-, sec- or tert-butylthio and so on.

“Haloalkyl” is an alkyl group substituted with halogen, preferably with fluoro, chloro and/or bromo, and there may be mentioned, for example, difluoromethyl, trifluoromethyl, 2-fluoroethyl, 2-chloroethyl, 2-bromoethyl, 2,2,2-trifluoroethyl, 2,2,2-trichloroethyl, 3-chloropropyl, 3-bromopropyl, 1-chloropropan-2-yl, 1-bromopropan-2-yl, 1,3-difluoropropan-2-yl, 2,3-dibromopropyl, 2,2-dichloro-3,3,3-trifluoropropyl and so on.

“Haloalkoxy” is an alkoxy group substituted with halogen, preferably with fluoro, chloro and/or bromo and there may be mentioned, for example, difluoromethoxy, trifluoromethoxy, 2-fluoroethoxy, 2-chloroethoxy, 2-bromoethoxy, 2,2,2-trifluoroethoxy, 2,2,2-trichloroethoxy, 3-chloropropoxy and so on.

“Haloalkylthio” is an alkylthio group substituted with halogen, preferably with fluoro, chloro and/or bromo, and there may be mentioned, for example, difluoromethylthio, trifluoromethylthio, 2-fluoroethylthio, 2-chloroethylthio, 2-bromoethylthio, 2,2,2-trifluoroethylthio, 2,2,2-trichloroethylthio, 3chloropropylthio and so on.

“Dialkylamino” is a dialkyl-substituted amino, whose alkyl moiety can be straight-chain or branched-chain, and there may be mentioned, for example, dimethylamino, diethylamino, di(n-propyl)amino, di(n-butyl) amino, methylethylamino, methyl(n-propyl)amino, methyl(iso-propyl)amino and so on.

Formula (I) provides a general definition of the triazolopyrimidines according to the invention. Preferred compounds of the formula (I) are those, in which

X represents chloro or bromo,

Y represents a hydrogen atom, chloro or bromo and

R represents phenyl-C₁₋₄ alkyl, optionally substituted by 1 to 5 identical or different radicals selected from fluoro, chloro, bromo, iodo C₁₋₄ alkyl, vinyl, dimethylamino, methoxy, methylcarbonyl, methoxycarbonyl, ethoxycarbonyl, methylthio, difluoromethyl, trifluoromethyl, C₁₋₂ fluoroalkoxy having 1 to 3 fluorine atoms, C₁₋₂ fluoroalkylthio having 1 to 3 fluorine atoms, phenyl, benzyl, phenoxy, cyano, nitro and 1,2,3-thiadiazol4-yl, or substituted by 1 radical selected from trimethylene and tetramethylene,

or

R represents diphenylmethyl, each of the phenyl groups being optionally substituted by 1 to 3 identical or different radicals selected from fluoro, chloro, bromo, iodo, C₁₋₄ alkyl, vinyl, dimethylamino, methylcarbonyl, methoxycarbonyl, ethoxycarbonyl, methoxy, methylthio, difluoromethyl, trifluoromethyl, C₁₋₂ fluoroalkoxy having 1 to 3 fluorine atoms, C₁₋₂ fluoroalkylthio having 1 to 3 fluorine atoms, phenyl, benzyl, phenoxy, cyano, nitro and 1,2,3-thiadiazol-4-yl, or being substituted by 1 radical selected from trimethylene and tetramethylene,

or

R represents naphthylmethyl, optionally substituted by 1 or 2 identical or different radicals selected from fluoro, chloro, bromo, iodo, C₁₋₄ alkyl, vinyl, dimethylamino, methylcarbonyl, methoxycarbonyl, ethoxycarbonyl, methoxy, methylthio, difluoromethyl, trifluoromethyl,: C₁₋₂ fluoroalkoxy having 1 to 3 fluorine atoms, C₁₋₂ fluoroalkylthio having 1 to 3 fluorine atoms, cyano and nitro,

or

R represents anthranylmethyl.

A preferred sub-group of the afore-mentioned group of compounds of the formula (I) are those, wherein

X represents chloro or bromo,

Y represents chloro or bromo and

R represents substituted phenyl-C₁₋₄ alkyl, optionally substituted diphenylmethyl or optionally substituted naphthylmethyl.

Particularly preferred are -those compounds of the formula (I), in which

X represents chloro or bromo,

Y represents a hydrogen atom, chloro or bromo and

R represents phenyl-C₁₋₄ alkyl, optionally substituted by 1 to 5 identical or different radicals selected from fluoro, chloro, bromo, iodo, C₁₋₄ alkyl, vinyl, dimethylamino, methoxy, methylcarbonyl, methoxycarbonyl, ethoxycarbonyl methylthio, difluoromethyl, trifluoromethyl, trifluoromethoxy, 2,2,2-trifluoroethoxy, trifluoromethylthio, 2,2,2-trifluoroethylthio phenyl, benzyl, phenoxy, cyano, nitro and 1,2,3-thiadiazol4-yl, or substituted by 1 radical selected from trimethylene and tetramethylene,

or

R represents diphenylmethyl, each of the phenyl groups being optionally substituted by 1 to 3 identical or different radicals selected from fluoro, chloro, bromo, C₁₋₄ alkyl, vinyl, dimethylamino, methoxy, methylcarbonyl, methoxycarbonyl, methylthio, difluoromethyl, trifluoromethyl, trifluoromethoxy, 2,2,2-trifluoroethoxy, trifluoromethylthio, 2,2,2-trifluoroethylthio, phenyl, benzyl phenoxy, cyano, nitro and 1,2,3-thiadiazol-4yl,

or

R represents naphthylmethyl, optionally substituted by 1 or 2 identical or different radicals selected from fluoro, chloro, bromo, C₁₋₄ alkyl, dimethylamino, methylcarbonyl, methoxycarbonyl, methoxy, methylthio, difluoromethyl, trifluoromethyl, trifluoromethoxy, trifluoromethylthio, cyano and nitro,

or

R represents anthranylmethyl.

A preferred sub-group of the afore-mentioned group of particularly preferred compounds are those, wherein

X is chloro or bromo,

Y is chloro or bromo and

R represents substituted phenyl-C₁₋₄ alkyl optionally substituted diphenylmethyl or optionally substituted naphthylmethyl.

Another preferred sub-group of the afore-mentioned group of particularly preferred compounds are those, wherein

X represents chloro,

Y represents a hydrogen atom or chloro and

R represents optionally substituted phenyl-C₁₋₄alkyl

Another preferred sub-group of the above-mentioned group of particularly preferred compounds are those, in which

X represents chloro,

Y represents chloro and

R represents phenyl-C₁₋₄ alkyl, which is substituted by 1 to 5 identical or different radicals of the group mentioned above.

If diethyl (4-chlorobenzyl)malonate and 3-amino-1,2,4-triazole are used as starting materials and phosphorus oxychloride is employed as halogenating agent, the process according to the invention can be illustrated by the following formula scheme.

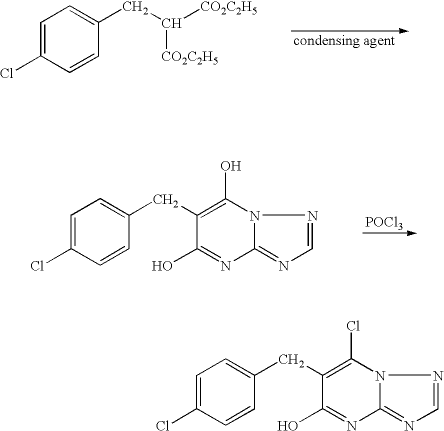

Formula (II) provides a general definition of the compounds, which are required as starting materials for carrying out the first step of the process according to the invention. In this formula, R preferably has those meanings, which have already been mentioned as preferred for this radical. R¹ preferably is methyl or ethyl, and R² preferably represents a hydrogen atom, methoxy or ethoxy.

The following compounds may be mentioned as examples of the compounds of the formula (II).

Diethyl benzylmalonate,

diethyl 4-chlorobenzylmalonate,

diethyl 4-methylbenzylmalonate,

diethyl 2-methoxybenzylmalonate,

diethyl 3-phenylpropylmalonate,

ethyl 2-formyl-3-phenylpropionate,

diethyl 1-naphthylmethylmalonate,

diethyl 4-nitrobenzylmalonate,

diethyl 4-trifluoromethylthiobenzylmalonate,

diethyl 3-trifluoromethylbenzylmalonate,

diethyl 4-cyanobenzylmalonate and so on.

The compounds of the formula (II) are known or can be prepared according to known processes (cf. “Modern Synthetic Reactions”, second edition, H. O. House, W. A. Benjamin, N C (1972), pages 510-570 and 734-765; “SHIN JIKKEN KAGAKU KOUZA” (New lecture on experimental chemistry) Vol. 15, Oxidation and Reduction II” p. 46, p. 62, p. 66, p. 72, p. 81, p. 86-88, p. 90, p. 109-110, p. 124, p. 185-186, p. 189, p. 192, p. 422-424 or p. 457 (published by Maruzen Ltd. on Feb. 20, 1977)).

Thus the compounds of the formula (II), in which R² represents C₁₋₄ alkoxy, can be prepared by a reacting compounds of the formula

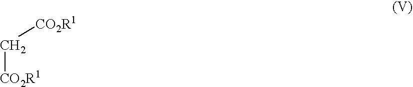

wherein

R¹ has the above-mentioned meanings,

with the compounds of the formula

R-Z (VI)

wherein

R has the above-mentioned meanings and

Z represents halogen,

or by

b) reducing compounds of the formula

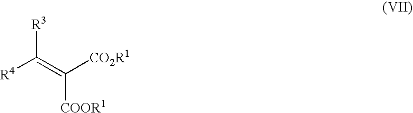

wherein

R¹ has the above-mentioned meanings,

R³ represents a hydrogen atom or C₁₋₃ alkyl and

R⁴ represents phenyl, optionally substituted by one or more radicals selected from halogen, alkyl, alkenyl, alkylene, dialkylamino, alkoxy, alkylcarbonyl, alkoxycarbonyl alkylthio, haloalkyl haloalkoxy, haloalkylthio, phenyl, benzyl, phenoxy, cyano, nitro and thiadiazolyl.

The compounds of the formulae (V) and (VI) are known or can be prepared by known processes.

The compounds of the formula (VII) are also known or can be prepared by known processes. Thus, they can be prepared by Knoevenagel Condensation of compounds of the formula

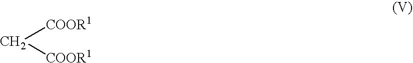

wherein

R¹ has the above-mentioned meanings,

with compounds of the formula

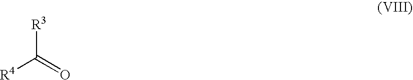

wherein

R³ and R⁴ have the above-mentioned meanings.

The compounds of the formula (VIII) are also known or can be prepared by known processes.

The compounds of the formula (II) in which R² represents a hydrogen atom, can be prepared by

c) reacting compounds of the formula

R—CH₂—COOR¹ (IX)

wherein

R and R¹ have the above-mentioned meanings,

with compounds of the formula

HCOOR¹ (X)

wherein

R¹ has the above-mentioned meanings.

The compounds of the formula (IX) and (X) are known or can be prepared by known processes.

Formula (III) provides a definition of the 3-amino-1,2,4-triazole, which is required as reaction component for carrying out the first step of the process according to the invention. The 3-amino-1,2,4-triazole is a known compound too.

Suitable diluents for conducting the first step of the process according to the invention are all customary inert organic solvents. The following can preferably be used: Aliphatic, alicyclic and aromatic hydrocarbons (which may optionally be chlorinated), for example, pentane, hexane, cyclohexane, petroleum ether, ligroine, benzene, toluene, xylene, dichloromethane, chloroform; carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; nitriles, for example, acetonitrile, priopionitrile, acrylonitrile etc.; esters, for example, ethyl acetate, amyl acetate etc.; acid amides, for example, dimethylformamide (DME), dimethylacetamide (DMA), N-methylpyrrolidone, 1,3-dimethyl-2-imidazolidinone, hexamethylphosphoric triamide (HMPA) etc.; sulfones and sulfoxides, for example, dimethyl sulfoxide (DMSO), sulfolane etc.; organic acids, for example, formic acid, acetic acid, trifluoroacetic acid, propionic acid etc.

Suitable acid-binding agents for conducting the first step of the process according to the invention are all customary inorganic and organic bases. The following can preferably be used: Inorganic bases, such as hydrides, hydroxides, carbonates, bicarbonates etc. of alkali metals or alkaline earth metals, for example, sodium hydride, lithium hydride, sodium hydrogen carbonate, potassium hydrogen carbonate, sodium carbonate, potassium carbonate, lithium hydroxice, sodium hydroxide, potassium hydroxide, calcium hydroxide etc.; inorganic alkali metal amides, for example, lithium amide, sodium amide, potassium amide etc.; and organic bases, such as, alcoholates, tertiary amines, dialkylaminoanilines and pyridines, for example, triethylamine, 1,1,4,4-tetramethylethylenediamine (TMEDA) N,N-dimethylaniline, N,N-diethylaniline, pyridine, 4-dimethylaminopyridine (DMAP), 1,4-diazabicyclo[2,2,2]octane (DABCO), 1,8-diazabicyclo[5,4,0]undec-7-ene (DBU) etc.; organolithium compounds, for example, methyl lithium, n-butyl lithium, sec-butyl lithium, phenyl lithium, dimethyl copper lithium, lithium diisopropylamide, lithium cyclohexylisopropylamide, lithium dicyclohexylamide, n-butyl lithium-DABCO, n-butyl lithium-TMEDA etc.

Upon using a compound of the formula (II), in which R² is a hydrogen atom, as a starting material, the first step of the process according to the invention can be conducted in the presence of an acid catalyst. Preferred acid catalysts are organic acids, such as formic acid, acetic acid, trifluoroacetic acid, propionic acid, methanesulfonic acid, benzenesulfonic acid, p-toluenesulfonic acid etc. Such acid catalysts can simultaneously be used as diluents for carrying out the reaction.

When carrying out the first step of the process according to the invention, the reaction temperatures can be varied within a substantially wide range. The reaction is generally carried out at a temperature between about 20° C. and about 200° C., preferably between about 80° C. and about 180° C.

The first step of the process according to the invention is generally carried out under atmospheric pressure but, if desired, can also be carried out under elevated or reduced pressure.

When carrying out the first step of the process according to the invention, in general 1 mole of a compound of the formula (II) is reacted with 0.9 to 1.2 moles of 3-amino-1,2,4triazole of the formula (III) in the presence of a diluent, such as dimethylformamide, and in the presence of an acid-binding agent, such as tri-n-butyl-amine.

Formula (IV) provides a general definition of the triazolopyrimidines, which are required as starting materials for carrying out the second step of the process according to the invention. In this formula, R preferably has those meanings, which have already been mentioned as preferred for this radical. Y¹ represents a hydrogen atom or a hydroxy group.

The triazolopyrimidines of the formula (IV) are obtained upon carrying out the first step of the process according to the invention.

Suitable reaction components for carrying out the second step of the process according to the invention are all customary halogenating agents. Preferred halogenating agents are phosphorus halides, such as phosphorus trichloride, phosphorus pentachloride and phosphorus tribromide, as well as phosphorus oxyhalides, such as phosphorus oxychloride, phosphorus oxybromide, phosgene, carbonyl bromide, oxalyl dichloride, thionyl chloride, thionyl bromide etc.

Suitable diluents for conducting the second step of the process according to the invention are all customary inert organic solvents. Preferred are aliphatic, alicyclic and aromatic hydrocarbons (which may optionally be chlorinated), for example, benzene, toluene, xylene, dichloromethane, chloroform, carbon tetrachloride, 1,2-dichloroethane, chlorobenzene, dichlorobenzene etc.; ethers, for example, diethylene glycol dimethyl ether (DGM) etc.; acid amides, for example, dimethylformamide (DMF), dimethylacetamide (DMA), N-methylpyrrolidone, 1,3-dimethyl-2-imidazolidinone, hexamethylphosphoric triamide (SPA) etc.

When carrying out the second step of the process according to the invention, the reaction temperatures can be varied within a substantially wide range. The reaction is generally carried out at a temperature between about 20° C. and about 180° C., preferably between about 40° C. and about 130° C.

The second step of the process according to the invention is generally carried out under atomospheric pressure but, if desired, can also be carried out under elevated pressure.

When carrying out the second step of the process according to the invention, in general 1 mole of a triazolopyrimidine of the formula (IV) is reacted with an excess amount of a halogenating agent, preferably in the presence of a catalytic amount of dimethylformamide.

In a particular variant, the process according to the invention can be conducted by reacting a compound of the formula (V) with a compound of the formula (VI) and then reacting the resulting compound of the formula (II) without isolation with 3-amino-1,2,4triazole of the formula (III).

Alternatively, the process according to the invention can also be conducted by reacting a compound of the formula (IX) with a compound of the formula (X) and then reacting the resulting compound of the formula (II) with 3-amino-1,2,4-triazole of the formula (III).

Upon conducting the process according to the invention, the triazolopyrimidines of the formula (IV) are generally isolated after the first step of the reaction, and then they are subjected to halogenation in the second step of the reaction.

The triazolopyrimidines of the formulae (IV) and (I) prepared by the process according to the invention can in each case be isolated from the reaction mixtures by customary procedures and can be purified by known methods, such as crystallization, chromatography etc.

The compounds according to the present invention exhibit a strong microbicidal activity. Thus, they can be used for combating undesired microorganisms, such as phytopathogenic fungi and bacteriae, in agriculture, horticulture and in the protection of materials. Undesirable microorganisms in the present case are to be understood as phytopathogenic fungi and bacteriae as well as fungi and bacteria destroying technical materials.

Generally, the compounds according to the invention can be used as fungicides for combating phytopathogenic fungi, such as Plasmodiophoromycetes, Oomycetes, Chytridiomycetes, Zygomycetes, Ascomycetes; Basdiomycetes and Deuteromycetes, and can also be used as bactericides for combating bacteriae, such as Pseudomonadaceae, Rhizobiaceae, Enterobacteriaceae, Corynebacteriaceae, Streptomycetaceae, Proteobacteriae and Gram-positive groups.

Some pathogens causing fungal diseases which come under the generic names listed above are mentioned as examples, but not by way of limitation:

Erwinia species, such as, for example, Erwinia amylovora;

Pythium species, such as, for example, Pythium ultimum;

Phytophthora species, such as, for example, Phytotophthora infestans;

Pseudoperonospora species, such as, for example, Pseudoperonospora humuli or

Pseudoperonospora cubensis;

Plasmopara species, such as, for example, Plasmopara viticola;

Bremia species, such as, for example, Bremia Lactucae;

Peronospora species, such as, for example, Peronospora pisi or P. brassicae;

Erysiphe species, such as, for example, Erysiphe graminis;

Sphaerotheca species, such as, for example, Sphaerotheca fuliginea;

Podosphaera species, such as, for example, Podosphaera leucotricha;

Venturia species, such as, for example, Venturi inaequalis;

Pyrenophora species, such as, for example, Pyrenophora teres or P. graminea (conidia form: Drechslera, syn: Helminthosporium);

Cochliobolus species, such as for example, Cochliobolus sativus (conidia form:

Drechslera, syn: Helminthosporium);

Uromyces species, such as, for example, Uromyces appendiculatus;

Puccinia species, such as, for example, Puccinia recondita;

Sclerotinia species, such as, for example, Sclerotinia sclerotiorum;

Tilletia species, such as, for example, Tilletia caries;

Ustilago species, such as, for example, Ustilago nuda or Ustilago avenae;

Pellicularia species, such as, for example, Pellicularia sasakii;

Pyricularia species, such as, for example, Pyricularia oryzae;

Fusarium species, such as, for example, Fusarium culmorum;

Botrytis species, such as, for example, Botrytis cinerea;

Septoria species, such as for example, Leptosphaeria nodorum;

Cercospora species, such as, for example, Cercospora canescens;

Alternaria species, such as, for example, Alternaria brassicae; and

Pseudocerosporella species, such as, for example, Pseudocerosporella herpotrichoides.

The compounds according to the invention are particularly suitable against infection of plants by pathogens, such as Botrytis cinerea, Pyricularia oryzae, Pellicularia sasakii, Alternaria mali Roberts Cochliobolus miyabeanus, Sphaerotheca fuliginea, Phytophthora infestans etc.

The fact that the active compounds are well tolerated by plants at the concentrations required for controlling plant diseases permits the treatment of above-ground parts of plants, of vegetative propagation stock and seeds, and of the soil.

Moreover, the active compounds, according to the present invention have a low toxicity against warm-blooded animals and therefore can be used safely.

In the protection of materials, the compounds according to the invention can be employed for protecting industrial materials against infection with and destruction by undesirable, microorganisms, such as fungi and bacteriae.

Industrial materials in the present context are understood as meaning non-living materials which have been prepared for use in industry. For example, industrial materials, which are intended to be protected by compounds according to the invention from microbial change or destruction, can be glues, sizes, paper and boards, textiles, leather, wood, paints and synthetic articles, cooling lubricants and other materials which can be infected with, or destroyed by, microorganisms. Parts of production plants, for example cooling—water circuits, which may be impaired by the multiplication of microorganisms, may also be mentioned within the scope of the materials to be protected. Industrial materials which may be mentioned within the scope of the present invention, are preferably glues, sizes, papers and boards, leather, wood, paints, cooling lubricants and heat—transfer liquids, specially preferably wood.

Examples of microorgnisms which are capable of bringing about degradation of, or change in, the industrial materials and which may be mentioned are bacteria, fungi, yeasts, algae and slime organisms. The compounds according to the invention preferably act against fungi, in particular moulds, wood—discolouring and wood—destroying fungi (Basidiomycetes) and against slime organisms and algae.

Microorganisms of the follwing genera may be mentioned by way of example:

Alternaria, such as Alternaria tenuis,

Aspergillus, such as Aspergillus niger,

Chaetomium, such as Chaetomium globosum,

Coniophora, such as Coniophora puetana,

Lentinus, such as Lentinus tigrinus,

Penicillium, such as Penicillium glaucum,

Polyporus, such as Polyporus versicolor,

Aureobasidium, such as Aureobasidium pullulans,

Sclerophoma, such as Sclerophoma pityophila,

Trichoderma, such as Trichoderma viride,

Escherichia, such as Escherichia coli,

Pseudomonas, such as Pseudomonas aeruginosa, and

Staphylococcus, such as Staphylococcus aureus.

The compounds according to the invention can be converted into the customary formulations, such as solutions, emulsions, wettable powders, suspensions, powders, foams, pastes, granules, tablets, aerosols, natural and synthetic materials impregnated with active compound, very fine capsules in polymeric substances, coating compositions for use on seed, and formulations used with burning equipment, such as fumigating cartridges, fumigating cans and fumigating coils, as well as ULV cold mist and warm mist formulations.

These formulations may be produced in known manner, for example by mixing the active compounds with extenders, that is to say liquid or liquefied gaseous or solid diluents or carriers, optionally with the use of surface-active agents, that is to say emulsifying agents and/or dispersing agents and/or foam-forming agents. In the case of the use of water as an extender, organic solvents can, for example, also be used as auxiliary solvents.

As liquid solvents diluents or carriers, there are suitable in the main, aromatic hydrocarbons such as xylene, toluene or alkyl naphthalenes, chlorinated aromatic or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol, as well as their ethers and esters, ketones, such as acetone, methyl ethyl ketone, methyl-isobutyl ketone or cyclohexanone, or strongly polar solvents, such as dimethylformamide and dimethylsulphoxide, as well as water. In case of using water as extender, for example, organic solvents can be used as auxiliary solvents.

By liquefied gaseous diluents or carriers are meant liquids which would be gaseous at normal temperature and under normal pressure, for example aerosol propellants, such as halogenated hydrocarbons as well as butane, propane, nitrogen and carbon dioxide.

As solid carriers there may be used ground natural minerals, such as kaolings, clays, talc, chalk, quartz, attapulgite, montmorillonite or diatomaceous earth, and ground synthetic minerals, such as highly-dispersed silicic acid, alumina and silicates. As solid carriers for granules there may be used crushed and fractionated natural rocks such as calcite, marble, pumice, sepiolite and dolomite, as well as synthetic granules of inorganic and organic meals, and granules of organic material such as sawdust, coconut shells, maize cobs and tobacco stalks.

As emulsifying and/or foam-forming agents there may be used non-ionic and anionic emulsifiers, such as polyoxyethylene-fatty acid esters, polyoxyethylene-fatty alcohol ethers, for example alkylaryl polyglycol ethers, alkyl sulphonates, alkyl sulphates, aryl sulphonates as well as albumin hydrolysis products.

Dispersing agents include, for example, lignin sulphite waste liquors and methylcellulose.

Adhesives such as carboxymethylcellulose and natural and synthetic polymers in the form of powders, granules or latices, such as gum arabic, polyvinyl alcohol and polyvinyl acetate, can be used in the formulations.

It is possible to use colorants such as inorganic pigments, for example iron oxide, titanium oxide and Prussian Blue, and organic dyestuffs, such as alizarin dyestuffs; azo dyestuffs or metal phthalocyanine dyestuffs, and trace nutrients, such as salts of iron, manganese, boron, copper, cobalt, molybdenum and zinc.

The formulations in general contain from 0.1 to 95 per cent by weight of active compound, preferably from 0.5 to 90 per cent by weight.

The active compounds according to the invention can be present in the formulations or in the various use forms as a mixture with other known active compounds, such as fungicides, bactericides, insecticides, acaricides, nematicides, herbicides, bird repellents, growth factors, plant nutrients and agents for improving soil structure.

In many cases, synergistic effects are achieved, i.e. the activity of the mixture exceeds the activity of the individual components.

Examples of co-components in mixtures are the following compounds:

Fungicides:

aldimorph, ampropylfos, ampropylfos potassium, andoprim, anilazine, azaconazole, azoxystrobin,

benalaxyl, benodanil, benomyl, benzamacril, benzamacril-isobutyl, bialaphos, binapacryl, biphenyl, bitertanol, blasticidin-S, bromuconazole, bupirimate, buthiobate,

calcium polysulphide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram, carpropamide,

debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,

edifenphos, epoxiconazole, etaconazole, ethirimol, etridiazole,

famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, fenhexamide,

guazatine,

hexachlorobenzene, hexaconazole, hymexazole,

imazalil, imibenconazole, iminoctadine, iminoctadine albesilate, iminoctadine triacetate, iodocarb, ipconazole, iprobenfos (IBP), iprodione, irumamycin, isoprothiolane, isovaledione, iprovalicarb,

kasugamycin, kresoxim-methyl, copper preparations, such as: copper hydroxide, copper naphthenate, copper oxychloride, copper sulphate, copper oxide, oxine-copper and Bordeaux mixture,

mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin,

nickel dimethyldithiocarbamate, nitrothal-isopropyl, nuarimol,

ofurace, oxadixyl, oxamocarb, oxolinic acid, oxycarboxim, oxyfenthiin,

paclobutrazole, pefurazoate, penconazole, pencycuron, phosdiphen, pimaricin, piperalin, polyoxin, polyoxorim, probenazole, prochloraz, procymidone, propamocarb, propanosine-sodium, propiconazole, propineb, pyrazophos, pyrifenox, pyrimethanil, pyroquilon, pyroxyfur,

quinconazole, quintozene (PCNB), quinoxyfen,

sulphur and sulphur preparations, spiroxamine,

tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, triflumizole, triforine, triticonazole, trifloxystrobine,

uniconazole,

validamycin A, vinclozolin, viniconazole,

zarilamide, zineb, ziram and also

Dagger G,

OK-8705,

OK-8801,

α-(1,1-dimethylethyl)-β-(2-phenoxyethyl)-1H-1,2,4triazole-1-ethanol,

α-(2,4-dichlorophenyl)-β-fluoro-β-propyl-1H-1,2,4triazole-1-ethanol,

α-(2,4-dichlorophenyl)-β-methoxy-α-methyl-1H-1,2,4triazole-1-ethanol,

α-(5-methyl-1,3-dioxan-5-yl)-β-[[4-(trifluoromethyl)-phenyl]-methylene]-1H-1,2,4-triazole-1-ethanol,

(5RS,6RS)-6-hydroxy-2,2,7,7-tetramethyl-5-(1H-1,2,4-triazol-1-yl)-3-octanone,

(E)-α-(methoxyimino)-N-methyl-2-phenoxy-phenylacetamide,

1-(2,4-dichlorophenyl)-2-(1H-1,2,4-triazol-1-yl)-ethanone O-(phenylmethyl)-oxime,

1-(2-methyl-1-naphthalenyl)-1H-pyrrol-2,5-dione,

1-(3,5-dichlorophenyl)-3-(2-propenyl)-2,5-pyrrolidinedione,

1-[(diiodomethyl)-sulphonyl]-4-methyl-benzene,

1-[[2-(2,4-dichlorophenyl)-1,3-dioxolan-2-yl]-methyl]-1H-imidazole,

1-[[2-(4-chlorphenyl)-3-phenyloxiranyl]-methyl]-1H-1,2,4-triazole,

1-[1-[2-[(2,4-dichlorophenyl)-methoxy]-phenyl]-ethenyl]-1H-imidazole,

1-methyl-5-nonyl-2-(phenylmethyl)-3-pyrrolidinole,

2′,6′-dibromo-2methyl-4′-trifluoromethoxy-4-trifluoro-methyl-1,3-thiazole-5-carboxanilide,

2,6-dichloro-5-(methylthio)-4-pyrimidinyl thiocyanate,

2,6-dichloro-N-(4-trifluoromethylbenzyl)-benzamide,

2,6-dichloro-N-[[4-(trifluoromethyl)-phenyl]-methyl]-benzamide,

2-(2,3,3-triiodo-2-propenyl)-2H-tetrazole,

2-[(1-methylethyl)-sulphonyl]-5-(trichloromethyl)-1,3,4-thiadiazole,

2-[[6-deoxy-4-O-(4-O-methyl-β-D-glycopyranosyl)-α-D-glucopyranosyl]-amino]-4-methoxy-1H-pyrrolo[2,3d]pyrimidine-5-carbonitrile,

-2-aminobutane,

2-bromo-2-(bromomethyl)-pentanedinitrile,

2-chloro-N-(2,3-dihydro-1,1,3-trimethyl-1H-inden-4-yl)-3-pyridinecarboxamide,

2-chloro-N-(2,6-dimethylphenyl)-N-(isothiocyanatomethyl)-acetamide,

2-phenylphenol (OPP),

3,4-dichloro-1-[4-(difluoromethoxy)-phenyl]-1H-pyrrol-2,5-dione,

3,5-dichloro-N-[cyano[(1-methyl-2-propinyl)-oxy]-methyl]-benzamide,

3-(1,1-dimethylpropyl-1-oxo-1H-indene-2-carbonitrile,

3-[2-(4-chlorophenyl)-5-ethoxy-3-isoxazolidinyl)-pyridine,

4chloro-2-cyano-N,N-dimethyl-5-(4-methylphenyl)-1H-imidazole-1-sulphonamide,

4-methyl-tetrazolo[1,5-a]quinazolin-5(4H)-one,

8-hydroxyquinoline sulphate,

9H-xanthene-2-[(phenylamino)-carbonyl]-9-carboxylic hydrazide,

bis-(1-methylethyl)-3-methyl-4-[(3-methylbenzoyl)-oxy]2,5-thiophenedicarboxylate,

cis-1-(4-chlorophenyl)-2-(1H-1,2,4-triazol-1-yl)-cycloheptanol,

cis-4-[3-[4-(1,1-dimethylpropyl)-phenyl-2-methylpropyl]-2,6-dimethyl-morpholine-hydrochloride,

ethyl-[(4-chlorophenyl)-azo]-cyanoacetate,

potassium hydrogen carbonate,

methanetetrathiol sodium salt,

methyl 1-(2,3-dihydro-2,2-dimethyl-1H-inden-1-yl)-1H-imidazole-5-carboxylate,

methyl N-(2,6-dimethylphenyl)-N-(5-isoxazolylcarbonyl)-DL-alaninate,

methyl N-(chloroacetyl)-N-(2,6-dimethylphenyl)-DL-alaninate,

N-(2,6-dimethylphenyl)-2-methoxy-N-(tetrahydro-2-oxo-3-furanyl)acetamide,

N-(2,6-dimethylphenyl)-2-methoxy-N-(tetrahydro-2-oxo-3-thienyl)-acetamide,

N-(2-chloro-4-nitrophenyl)-4-methyl-3-nitro-benzenesulphonamide,

N-(4-cyclohexylphenyl)-1,4,5,6-tetrahydro-2-pyrimidineamine,

N-(4-hexylphenyl)-1,4,5,6-tetrahydro-2-pyrimidineamine,

N-(5-chloro-2-methylphenyl)-2-methoxy-N-(2-oxo-3-oxazolidinyl)-acetamide,

N-(6-methoxy)-3-pyridinyl)-cyclopropanecarboxamide,

N-[2,2,2-trichloro-1-[(chloroacetyl)-amino]-ethyl]-benzamide,

N-[3-chloro4,5-bis(2-propinyloxy)-phenyl]-N′-methoxy-methanimidamide,

N-formyl-N-hydroxy-DL-alanine-sodium salt,

O,O-diethyl [2-(dipropylamino)-2-oxoethyl]-ethylphosphoramidothioate,

O-methyl S-phenyl phenylpropylphosphoramidothioate,

S-methyl 1,2,3-benzothiadiazole-7-carbothioate,

spiro[2H]-1-benzopyran-2,1′(3′H)-isobenzofuran]-3′-one,

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/acaricides/nematicides:

abamectin, acephate, acetamiprid, acrinathrin, alanycarb, aldicarb, aldoxycarb, alphacypermethrin, alphamethrin, amitraz, a%ermectin, AZ 60541, azadirachtin, azamethiphos, azinphos A, azinphos M, azocyclotin,

Bacillus popilliae, Bacillus sphaericus, Bacillus subtilis, Bacillus thuringiensis, baculoviruses, Beauveria bassiana, Beauveria tenella, bendiocarb, benfuracarb, bensultap, benzoximate, betacyfluthrin, bifenazate, bifenthrin, bioethanomethrin, biopermethrin, BPMC, bromophos A, bufencarb, buprofezin, butathiofos, butocarboxim, butylpyridaben,

cadusafos, carbaryl, carbofuran, carbophenothion, carbosulfan, cartap, chloethocarb, chlorethoxyfos, chlorfenapyr, chlorfenviphos, chlorfluazuron, chlormephos, chlorpyrifos, chlorpyrifos M, chlovaporthrin, cis-resmethrin, cispermethrin, clocythrin, cloethocarb, clofentezine, cyanophos, cycloprene, cycloprothrin, cyfluthrin, cyhalothrin, cyhexatin, cypermethrin, cyromazine,

deltamethrin, demeton M, demeton S, demeton-S-methyl, diafenthiuron, diazinon, dichlorvos, diflubenzuron, dimethoat, dimethylvinphos, diofenolan, disulfoton, docusat-sodium, dofenapyn,

eflusilanate, emamectin, empenthrin, endosulfan, Entomopfthora spp., esfenvalerate, ethiofencarb, ethion, ethoprophos, etofenprox, etoxazole, etrimphos,

fenamiphos, fenazaquin, fenbutatin oxide, fenitrothion, fenothiocarb, fenoxacrim, fenoxycarb, fenpropathrin, fenpyrad, fenpyrithirn, fenpyroximate, fenvalerate, fipronil, fluazuron, flubrocythrinate, flucycloxuron, flucythrinate, flufenoxuron, flutenzine, fluvalinate, fonophos, fosmethilan, fosthiazate, fubfenprox, furathiocarb,

granulosis viruses,

halofenozide, HCH, heptenophos, hexaflumuron, hexythiazox, hydroprene,

imidacloprid, isazophos, isofenphos, isoxathion, ivermectin,

lambda-cyhalothrin, lufenuron,

malathion, mecarbam, metaldehyde, methamidophos, Metharhizium anisopliae, Metharhizium flavoviride, methidathion, methiocarb, methomyl, methoxyfenozide, metolcarb, metoxadiazone, mevinphos, milbemectin, monocrotophos,

naled, nitenpyram, nithiazine, novaluron, nuclear polyhedrosis viruses,

omethoat, oxamyl, oxydemethon M,

Paecilomyces fumosoroseus, parathion A, parathion M, permethrin, phenthoat, phorat, phosalone, phosmet, phosphamidon, phoxim, pirimicarb, pirimiphos A, pirimiphos M, profenofos, promecarb, propoxur, prothiofos, prothoat, pymetrozine, pyraclofos, pyresmethrin, pyrethrum, pyridaben, pyridathion, pyimidifen, pyriproxyfen,

quinalphos,

ribavirin,

salithion, sebufos, silafluofen, spinosad, sulfotep, sulprofos,

tau-fluvalinate, tebufenozide, tebufenpyrad, tebupirimiphos, teflubenzuron, tefluthrin, temephos, temivinphos, terbufos, tetrachlorvinphos, theta-cypermethrin, thiamethoxam, thiapronil, thiatriphos, thiocyclam hydrogen oxalate, thiodicarb, thiofanox, thuringiensin, tralocythrin, tralomethrin, triarathene, triazamate, triazophos, triazuron, trichlophenidine, trichlorfon, triflumuron, trimethacarb, thiacloprid,

vamidothion, vaniliprole, Verticillium lecanii,

YI 5302,

zeta-cypermethrin, zolaprofos,

(1R-cis)-[5-(phenylmethyl)-3-furanyl]-methyl-3-[(dihydro-2-oxo-3(2H )-furanylidene)-methyl]2,2-dimethylcyclopropanecarboxylate,

(3-phenoxyphenyl)-methyl 2,2,3,3-tetramethylcyclopropanecarboxylate,

1-[(2-chloro-5-thiazolyl)methyl]tetrahydro-3,5dimethyl-.N-nitro-1,3,5-triazine-2(1H)-imine,

2-(2-chloro-6-fluorophenyl)4-[4-(1,1dimethylethyl)phenyl]4,5-dihydro-oxazole,

2-(acetyloxy)-3dodecyl-1,4naphthalenedione,

2-chloro-N-[[[4(1-phenylethoxy)-phenyl]-amino]-carbonyl]-benzamide,

2-chloro-N-[[[4-(2,2-dichloro-1,1-difluoroethoxy)-phenyl]-amino]-carbonyl]-benzamide,

3-methylphenyl propylcarbamate

4-[4-(4-ethoxyphenyl)4-methylpentyl]-1-fluoro-2-phenoxy-benzene,

4-chloro-2-(1,1-dimethylethyl)-5-[[2-(2,6-dimethyl-4-phenoxyphenoxy)ethyl]thio]-3(2H)-pyridazinone,

4-chloro-2-(2-chloro-2-methylpropyl)-5-[(6-iodo-3-pyridinyl)methoxy]-3(2H)-pyridazinone,

4-chloro-5-[(6-chloro-3-pyridinyl)methoxy]-2-(3,4-dichlorophenyl)-3(2H)-pyridazinone,

Bacillus thuringiensis strain EG-2348,

[2-benzoyl-1-(1,1-dimethylethyl)-hydrazinobenzoic acid,

-2,2-dimethyl-3-(2,4-dichlorophenyl)-2-oxo-1-oxaspiro[4.5]dec-3-en-4-yl butanoate,

[3-[(6-chloro-3-pyridinyl)methyl]-2-thiazolidinylidene]-cyanamide,

dihydro-2-(nitromethylene)-2H-1,3-thiazine-3(4H)-carboxaldehyde,

ethyl [2-[[1,6-dihydro-6-oxo-1-(phenylmethyl)-4-pyridazinyl]oxy]ethyl]-carbamate,

N-(3,4,4-trifluoro-1-oxo-3-butenyl)-glycine,

N-(4-chlorophenyl)-3-[4-(difluoromethoxy)phenyl]-4,5-dihydro-4-phenyl-1H-pyrazole-1-carboxamide,

N-[(2-chloro-5-thiazolyl)methyl]-N′-methyl-N″-nitro-guanidine,

N-methyl-N′-(1-methyl-2-propenyl)-1,2-hydrazinedicarbothioamide,

N-methyl-N′-2-propenyl-1,2-hydrazinedicarbothioamide,

O,O-diethyl [2-(dipropylamino)-2-oxoethyl]-ethylphosphoramidothioate.

The active compounds can be used as such or in the form of their formulations or the use forms prepared therefrom by further dilution, such as ready-to-use solutions, emulsions, suspensions, powders, tablets, pastes, microcapsules and granules. They are used in the customary manner, for example by watering, immersion, spraying, atomising, misting, vaporizing, injecting, forming a slurry, brushing on, dusting, scattering, dry dressing, moist dressing, wet dressing, slurry dressing or encrusting.

In the treatment of parts of plants, the active compounds concentration in the use forms can be varied within a substantial range. They are, in general, from 1 to 0.0001% by weight, preferably from 0.5 and 0.001%.

For the treatment of seed, amounts of active compound of 0.001 to 50 g, especially 0.01 to 10 g, are generally employed per kilogram of seed.

For the treatment of soil, active compound concentrations, at the point of action, of 0.00001 to 0.1% by weight, especially of 0.0001 to 0.02%, are generally employed.

As already mentioned above, all plants and parts of plants can be treated according to the invention. In a preferred embodiment naturally occurring plant species and plant varieties or those obtained by conventional biological breeding methods, such as crossbreeding or protoplast fusion as well as parts of such plants are treated. In an additional preferred embodiment transgenic plants and plant varieties which have been obtained by genetic engineering methods, possibly in combination with conventional methods (genetically modified organisms) and parts of such plants are treated. The term “parts” or “parts of plants” or “plant parts” is explained above.

According to the invention plants of the plant varieties commercially available or used at any particular time are very preferably treated. Plant varieties are understood to be plants with specific properties (“traits”) which have been obtained both by conventional breeding, by mutagenesis or by recombinant DNA techniques. They can be varieties, biotypes or genotypes.

Depending on the species or varieties of plants, their location and growth conditions (the types of soil, climate, vegetation period and feed concerned), superadditive (“synergistic”) effects can occur as a result of the treatment according to the invention. Effects such as for example reduced application rates and/or broadening of the activity spectra and/or increased activity of the compounds and compositions usable according to the invention, improved plant growth, increased tolerance of high or low temperatures, increased tolerance of dry conditions or water or ground salt contents, increased flowering capacity, facilitated harvesting, acceleration of maturity, increased crop yields, higher quality and/or increased nutritional value of the harvested crops and increased storing quality and/or processibility of the harvested crops are possible, which are greater than those actually expected.

Preferred transgenic plants or plant varieties (obtained by genetic engineering) to be treated according to the invention include all plants which as a result of the genetic modification concerned have received genetic material which provides them with particularly advantageous valuable properties (“traits”). Examples of such properties are improved plant growth, increased tolerance of high or low temperatures, increased tolerance of dry conditions or water or ground salt contents, increased flowering capacity, facilitated harvesting, acceleration of maturity, increased crop yields, higher quality and/or increased nutritional value of the harvested crops and increased storing quality and/or processibility of the harvested crops. Additional and particularly noteworthy examples of such properties are increased resistance of the plants to animal and microbial pests, such as to insects, mites, phytopathogenic fungi, bacteria and/or viruses as well as increased tolerance by the plants of certain herbicidal active compounds. Examples which may be mentioned of transgenic plants are the important crop plants such as cereals (wheat and rice), corn, soybeans, potatoes, cotton, rape and fruit plants (producing apples, pears, citrus fruits and grapes), the crop plants corn, soybeans, potatoes, cotton and rape being particularly noteworthy. Particularyl significant properties (“traits”) are increased resistance of the plants to insects due to the toxins forming in the plants, and in particular those which are produced in the plants (hereinafter referred to as “Bt plants”) by the genetic material obtained from Bacillus Thuringiensis (e.g. by the genes Cry1A(a), Cry1A(b), Cry1A(c), Cry11A, CryIIIB2, Cry9c Cry2Ab, Cry3Bb and CryIF and combinations thereof). Particularly significant properties (“traits”) are the increased resistance of plants to fungi, bacteria and viruses due to systemically acquired resistance (SAR), systemin, phytoalexins, elicitors and resistance genes and correspondingly expressed proteins and toxins. Particulary significant properties (“traits”) are also increased tolerance by the plants of certain herbicidal active compounds, such as for example imidazolinones, sulphonylureas, glyphosate or phosphinotricine (e.g. the “PAT” gene). The corresponding genes imparting the required properties (“traits”) can also occur in the transgenic plants in combination with each other. Examples which may be mentioned of “Bt plants” are varieties of corn, cotton, soybeans and potatoes which are sold under the trade names YIELD GARD® (e.g. corn, cotton, soybeans), KnockOut® (e.g. corn), StarLink® (e.g. corn), Bollgard® (cotton), Nucotn® (cotton) and NewLeaf® (potatoes). Examples which may be mentioned of herbicide-tolerant plants are varieties of corn, cotton and soybeans which are sold under the trade names Roundup Ready® (tolerance of glyphosate, e.g. corn, cotton, soybeans), Liberty Link® (tolerance of phosphinotricine, e.g. rape), IMI® (tolerance of imidazolinones) and STS®D (tolerance of sulphonylureas, e.g. corn). Herbicide-resistant plants (bred for herbicide tolerance in the conventional manner) which may be mentioned are also the varieties (e.g. corn) sold under the name Clearfield®.; The above statements do of course also apply to any plant varieties which may be developed in the future or launched onto the market in the future and which have the genetic properties (“traits”) described above or developed in the future.

According to the invention the abovementioned plants can be particularly advantageously treated with the compounds of the general formula I or the active compound mixtures according to the invention. The preferred ranges mentioned above for the active compounds or mixtures also apply to the treatment of these plants. Particularly advantageous is the treatment of plants with the compounds or mixtures specifically listed in the present text.

The preparation and the use of the compounds according to the invention is illustrated by the following examples. The invention, however, is not limited to said examples in any way.

### SYNTHESIS EXAMPLE 1

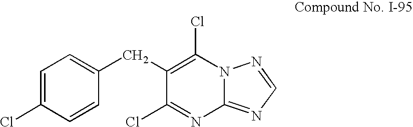

60% Sodium hydride (4.0 g) was added to a solution of diethyl malonate (16.0 g) in N,N-dimethylformamide (50 ml) whilst stirring at room temperature. After stirring for one additional hour at room temperature, 4-chlorobenzyl chloride (16.1 g) was added to the reaction mixture, which then was stirred at room temperature for further 3 hours. The reaction mixture was then diluted with water and acidified with concentrated hydrochloric acid, and the resulting mixture was extracted, with ethyl acetate. The organic phase was washed with water, dried over anhydrous magnesium sulfate and concentrated under reduced pressure. 3-Amino-1,2,4-triazole (8.4 g) and tri-n-butylamine (18.5 g) were added to the residue obtained, and the resulting mixture was stirred at 170° C. for 5 hours. After cooling, a solid product was obtained, which was washed with water and dried. N,N-Dimethylformamide (0.8 g) was then added to a suspension of said solid product (26.7 g) in phosphorus oxychloride (61.3 g), and the resulting mixture was first stirred at room temperature for 30 minutes and then heated under reflux for 6 hours. After cooling to room temperature again, the reaction mixture was added to water and the resulting mixture was extracted with ethyl acetate. The combined organic phases were washed with water, dried over anhydrous magnesium sulfate and then concentrated under reduced pressure. The remaining residue was purified by silica gel column chromatography (eluent ethyl acetate:hexane 1:1) to obtain 5,7-dichloro-6-(4-chlorobenzyl)[1,2,4]triazolo[1,5-a]pyrimidine (8.0 g).

mp 165-167° C.

### SYNTHESIS EXAMPLE 2

First Step

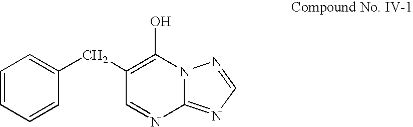

A solution of ethyl 3-phenylpropionate (3.0 g) and methyl formate (17.2 g) in N,N-dimethylformamide (13 ml) was added dropwise to a suspension of 60% sodium hydride (1.4 g) in N,N-dimethylformamide (18 ml) at room temperature. After stirring the reaction mixture for 15 hours at room temperature, it was poured into water and acidified with hydrochloric acid. The mixture was then extracted with ethyl acetate, and the combined organic phases were washed with water, dried over anhydrous magnesium sulfate and then concentrated under reduced pressure 3-Amino-1,2,4-triazole (1.42 g) and acetic, acid (55 ml) were added to the residue obtained before, and the mixture was heated under reflux for 6 hours. The reaction mixture was poured into a mixture of water and hexane. The crystals formed were filtered off, washed with water and then with hexane and dried to: obtain 6-benzyl[1,2,4]triazolo[1,5-a]pyrimidin-7-ol (0.9 g).

IR (KBr) ν: 1608.1, 1530.0, 1478.6, 1453.9, 1366.2, 1264.1, 700.0 cm⁻¹.

Second Step

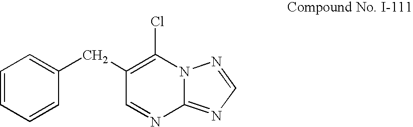

N,N-Dimethylformamide (0.1 ml) was added to a mixture of 6-benzyl[1,2,4]triazolo[1,5a]pyrimidin-7-ol (08. g) and phosphorus oxychloride (10 ml), and the resulting mixture was heated under reflux for 6 hours. The reaction mixture was then concentrated under reduced pressure and the remaining residue was dissolved in dichloromethane. The solution was poured into ice water. The resulting mixture was brought to basic reaction by adding a 40% aqueous solution of sodium hydroxide, and then it was extracted with dichloromethane. The combined organic phases were washed with water, dried over anhydrous magnesium sulfate and concentrated under reduced pressure. The residue was purified by silica gel column chromatography (eluent:dichloromethane) to obtain 6-benzyl-7-chloro[1,2,4]triazolo[1,5-a]pyrimidine (0.8 g).

mp 123-127° C.

The following Tables 1 and 2 show compounds according to the invention, which were synthesized in a similar manner as the compounds of Synthesis Examples 1 and 2. Said compounds of Synthesis Examples 1 and 2 are also listed in Table 1.

TABLE 1

| | |
|---|---|
| | (Ia) |
| 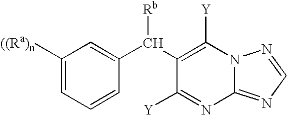 | |

| Compound | | | | | Melting |
|---|---|---|---|---|---|
| No. | X | Y | (R^{a})n | R^{b} | point (° C.) |
| I-1 | Cl | Cl | 2,3-(CH₃)₂ | H | |
| I-2 | Cl | Cl | 2,3,4,5,6-(CH₃)₅ | H | |
| I-3 | Cl | Cl | 2,3,4,5,6-Cl₅ | H | |
| I-4 | Cl | Cl | 2,3,4,5,6-F₅ | H | |
| I-5 | Cl | Cl | 2,3,4,5-Cl₄, 6-CF₃ | H | |
| I-6 | Cl | Cl | 2,3,4-F₃ | H | 205-208 |
| I-7 | Cl | Cl | 2,3,5,6-(CH₃)₄ | H | |
| I-8 | Cl | Cl | 2,3,5,6-F₄, 4-CF₃ | H | |
| I-9 | Cl | Cl | 2,3,5,6-F₄, 4-OCH₃ | H | |
| I-10 | Cl | Cl | 2,3,6-Cl₃ | H | 167-170 |
| I-11 | Cl | Cl | 2,3,6-F₃ | H | |
| I-12 | Cl | Cl | 2,3-Cl₂ | H | 214-216 |
| I-13 | Cl | Cl | 2,3-F₂ | H | |
| I-14 | Cl | Cl | 2,4-(CF₃)₂ | H | 157-158 |
| I-15 | Cl | Cl | 2,4-(CH₃)₂ | H | 160-163 |
| I-16 | Cl | Cl | 2,4-(CH₃)₂ | CH₃ | 155-156 |
| I-17 | Cl | Cl | 2,3-(OCH₃)₂ | H | |
| I-18 | Cl | Cl | 2,4,5-F₃ | H | 152-154 |
| I-19 | Cl | Cl | 2,4,6-(CH₃)₃ | H | 166-167 |
| I-20 | Cl | Cl | 2,4,6-F₃ | H | 142-148 |
| I-21 | Cl | Cl | 2,4-Cl₂ | H | 203-204 |
| I-22 | Cl | Cl | 2,4-F₂ | H | 183-185 |
| I-23 | Cl | Cl | 2,5-(CF₃)₂ | H | 155-156 |
| I-24 | Cl | Cl | 2,5-(CH₃)₂ | H | |
| I-25 | Cl | Cl | 2,5-Cl₂ | H | 216-217 |
| I-26 | Cl | Cl | 2,5-F₂ | H | |
| I-27 | Cl | Cl | 2,6-(CH₃)₂ | H | |
| I-28 | Cl | CI | 2,6-Cl₂ | H | 161-163 |
| I-29 | Cl | Cl | 2,6-F₂ | H | 164-167 |
| I-30 | Cl | Cl | 2-Br | H | 184-185 |
| I-31 | Cl | Cl | 2-Br, 5-F | H | |
| I-32 | Cl | Cl | 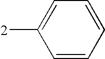 | H | 151-152 |
| I-33 | Cl | Cl | 2-CF₃, 4-F | H | 180-181 |
| I-34 | Cl | Cl | 2-CF₃ | H | |
| I-35 | Cl | Cl | 2-CH₃ | H | 141-142 |
| I-36 | Cl | Cl | 2-CH₃ | CH₃ | 131-136 |
| I-37 | Cl | Cl | 2-CH₃, 3-NO₂ | H | |
| I-38 | Cl | Cl | 2-Cl | H | 177-180 |
| I-39 | Br | Br | 2-Cl | H | |
| I-40 | Cl | Cl | 2-Cl | CH₃ | 133-134 |
| I-41 | Cl | Cl | 2-Cl, 4-F | H | 203-204 |
| I-42 | Cl | Cl | 2-Cl, 6-F | H | 152-155 |
| I-43 | Cl | Cl | 2-Cl, 6-OCHF₂ | H | |
| I-44 | Cl | Cl | 2,5-(OCH₃)₂ | H | |
| I-45 | Cl | Cl | 2-F | H | |
| I-46 | Cl | Cl | 2-F, 3-CH₃ | H | |
| I-47 | Cl | Cl | 2-F, 3-Cl | H | |
| I-48 | Cl | Cl | 2-F, 4-Br | H | |
| I-49 | Cl | Cl | 2-F, 4-Cl | H | |
| I-50 | Cl | Cl | 2-I | H | |
| I-51 | Br | Br | 4-OCF₃ | H | |
| I-52 | Cl | Cl | 3,4-Br₂ | H | |
| I-53 | Br | Br | 4-CF₃ | H | |
| I-54 | Cl | Cl | 4-CO₂CH₃ | H | 159-164 |
| I-55 | Cl | Cl | 4-COCH₃ | H | |
| I-56 | Cl | Cl | 2-OCH₃ | H | 135-137 |
| I-57 | Cl | Cl | 2-OCH_{3, 5-NO}₂ | H | |
| I-58 | Cl | Cl | 2-SCH₂CF₃ | H | |
| I-59 | Cl | Cl | 3,4-(CH₂)₄— | H | |
| I-60 | Cl | Cl | 3,4-(CH₃)₂ | H | |
| I-61 | Cl | Cl | 3,4,5-(OCH₃)₃ | H | |
| I-62 | Cl | Cl | 3,4-Cl₂ | H | 181-183 |
| I-63 | Cl | Cl | 3,4-F₂ | H | |
| I-64 | Cl | Cl | 3,5-(CF₃)₂ | H | 166-168 |
| I-65 | Cl | Cl | 3,5-(CH₃)₂ | H | |
| I-66 | Cl | Cl | 3,4-(OCH₃)₂ | H | |
| I-67 | Cl | Cl | 3,5-(OCH₃)₂ | H | |
| I-68 | Cl | Cl | 3,5-Br₂ | H | |
| I-69 | Cl | Cl | 3,5-Cl₂ | H | |
| I-70 | Cl | Cl | 3,5-F₂ | H | |
| I-71 | Cl | Cl | 3-Br | H | 150-152 |
| I-72 | Cl | Cl | 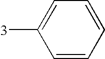 | H | |
| I-73 | Cl | Cl | 3-CF₃ | H | 160-161 |
| I-74 | Cl | Cl | 3-CH═CH₂ | H | |
| I-75 | Cl | Cl | 3-CH₃ | H | 128-129 |
| I-76 | Cl | Cl | 3-Cl | H | 125-127 |
| I-77 | Br | Br | 3-Cl | H | |
| I-78 | Cl | Cl | 2-CN | H | 180-183 |
| I-79 | Cl | Cl | 3-F | H | |
| I-80 | Cl | Cl | 3-I | H | |
| I-81 | Cl | Cl | 3-NO₂ | H | |
| I-82 | Cl | Cl | 4-OCHF₂ | H | |
| I-83 | Cl | Cl | 3-OCF₃ | H | |
| I-84 | Cl | Cl | 3-OCHF₂ | H | |
| I-85 | Cl | Cl | 3-OCH₂CF₃ | H | |
| I-86 | Cl | Cl | 3-OCH₃ | H | 134-135 |
| I-87 | Cl | Cl | 3-OCHF₂, 4-Cl, | H | |
| I-88 | Cl | Cl | 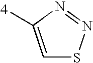 | H | |
| I-89 | Cl | Cl | 4-Br | H | 183-184 |
| I-90 | Cl | Cl | 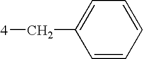 | H | |
| I-91 | Cl | Cl | 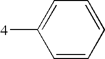 | H | |
| I-92 | Cl | Cl | 4-CF₃ | H | 193-194 |
| I-93 | Cl | Cl | 4-CH═CH₂ | H | |
| I-94 | Cl | Cl | 4-CH₃ | H | 143-144 |
| I-95 | Cl | Cl | 4-Cl | H | 165-167 |
| I-96 | Br | Br | 4-Cl | H | |
| I-97 | Cl | Cl | 4-Cl | CH₃ | 104-105 |
| I-98 | Cl | Cl | 4-Cl | C₃H₇-n | |
| I-99 | Cl | Cl | 4-CN | H | 231-233 |
| I-100 | Cl | Cl | 4-F | H | 157-158 |
| I-101 | Cl | Cl | 4-C₃H₇-iso | H | |
| I-102 | Cl | Cl | 4-SCHF₂ | H | |
| I-103 | Cl | Cl | 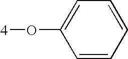 | H | |
| I-104 | Cl | Cl | 4-OCF₃ | H | 118-121 |
| I-105 | Cl | Cl | 4-OCH₃ | H | 140-142 |
| I-106 | Cl | Cl | 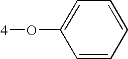 | H | |
| I-107 | Cl | Cl | 4-SCF₃ | H | 147-148 |
| I-108 | Cl | Cl | 4-SCH₃ | H | |
| I-109 | Cl | Cl | 4-C₄H₉-tert | H | |
| I-110 | Cl | Cl | H | H | 118-120 |
| I-111 | Cl | H | H | H | 123-127 |
| I-112 | Cl | Cl | H | CH₃ | 113-118 |
| I-113 | Cl | Cl | H | C₂H₅ | 113-114 |
| I-114 | Cl | Cl | H | 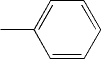 | 174-175 |
| I-115 | Cl | Cl | 4-Cl | 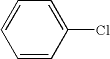 | |
| I-116 | Br | Br | 4-SCF₃ | H | |
| I-117 | Cl | Cl | 4-CH₃ | 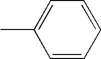 | |
| I-118 | Cl | Cl | 4-Cl | 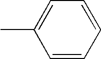 | |
| I-119 | Cl | Cl | 4-OCH₃ | 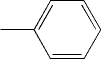 | |
| I-120 | Cl | Cl | 4-F | 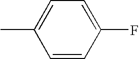 | 155-160 |
| I-121 | Cl | H | 2-Cl | H | 118-120 |
| I-122 | Cl | H | 3-Cl | H | 107-109 |
| I-123 | Cl | H | 4-Cl | H | 192-193 |
| I-124 | Cl | H | 2-CH₃ | H | 145-146 |
| I-125 | Cl | H | 3-CH₃ | H | 82-85 |
| I-126 | Cl | H | 4-CH₃ | H | 122-125 |
| I-127 | Cl | H | 4-CHF₂ | H | 128-132 |
| I-128 | Cl | H | 2-OCH₃ | H | |
| I-129 | Cl | H | 3-OCH₃ | H | 67-68 |
| I-130 | Cl | H | 4-OCH₃ | H | |
| I-131 | Cl | H | 4-OCF₃ | H | 67-70 |
| I-132 | Cl | H | 2-Br | H | |
| I-133 | Cl | H | 3-Br | H | |
| I-134 | Cl | H | 4-Br | H | 129-130 |
| I-135 | CI | H | 2-F | H | |
| I-136 | Cl | H | 3-F | H | |
| I-137 | Cl | H | 4-F | H | 166-167 |
| I-138 | Cl | H | 2-CF₃ | H | 166-170 |
| I-139 | Cl | H | 3-CF₃ | H | 99-102 |
| I-140 | Cl | H | 4-CF₃ | H | 135-136 |
| I-141 | Cl | H | 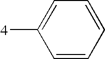 | 114-117 | |
| I-142 | Cl | H | 2,3-Cl₂ | H | 134-135 |
| I-143 | Cl | H | 2,4-Cl₂ | H | 148-150 |
| I-144 | Cl | H | 2,5-Cl₂ | H | |
| I-145 | Cl | H | 2,6-Cl₂ | H | 179-181 |
| I-146 | Cl | H | 3,4-Cl₂ | H | 138-143 |
| I-147 | Cl | H | 2,5-(CF₃)₂ | H | |
| I-148 | Cl | H | 3,5-(CF₃)₂ | H | |
| I-149 | Cl | H | 4-C₃H₇-iso | H | |
| I-150 | Cl | H | 3-NO₂ | H | |
| I-151 | Cl | H | 4-N(CH₃)₂ | H | |
| I-152 | Cl | H | 3,5-Cl₂ | H | |
| I-153 | Cl | H | 3,5-F₂ | H | |
| I-154 | Cl | H | 2,3-F₂ | H | |
| I-155 | Cl | H | 2,4-F₂ | H | |
| I-156 | Cl | H | 2,5-F₂ | H | |
| I-157 | CI | H | 2,6-F₂ | H | |
| I-158 | Cl | H | 3,4-F₂ | H | |
| I-159 | Cl | H | 3,5-Br_{2 H} | | |
| I-160 | Cl | H | 3,4-Br_{2 H} | | |
| I-161 | Cl | H | 2,3-(CH₃)₂ | H | |
| I-162 | Cl | H | 2,4-(CH₃)₂ | H | |
| I-163 | Cl | W | 2,5-(CH₃)₂ | H | |
| I-164 | Cl | H | 2,6-(CH₃)₂ | H | |
| I-165 | Cl | H | 3,4-(CH₃)₂ | H | |
| I-166 | Cl | H | 3,5-(CH₃)₂ | H | |
| I-167 | Cl | H | 2,3-(OCH₃)₂ | H | |
| I-168 | Cl | H | 2,4-(OCH₃)₂ | H | |
| I-169 | Cl | H | 2,5-(OCH₃)₂ | H | |
| I-170 | Cl | H | 3,4-(OCH₃)₂ | H | |

TABLE 2

| | |
|---|---|
| | (Ib) |
| 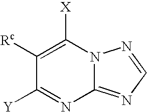 | |

| Compound | | | | Melting |
|---|---|---|---|---|
| No. | X | Y | R^{c} | point (° C.) |
| I-171 | Cl | Cl | 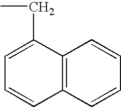 | |
| I-172 | Cl | Cl | 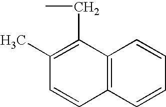 | |
| I-173 | Cl | Cl | 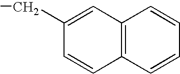 | |
| I-174 | Cl | Cl | 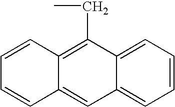 | |
| I-175 | Cl | Cl | 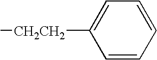 | |
| I-176 | Cl | Cl | 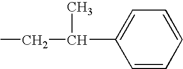 | |
| I-177 | Cl | Cl | 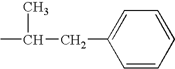 | 126-127 |
| I-178 | Cl | Cl | 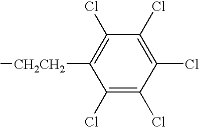 | >250 |

### BIOLOGICAL TEST EXAMPLES

### TEST EXAMPLE A

Test of foliar spray effect against Botrytis cinera

Preparation of Formulations of the Compounds Tested

Active compound: 30-40 parts by weight

Carrier: mixture of diatomaceous earth and kaolin (i:5), 55-65 parts by weight

Emulsifier: p1%yoxyethylene alkyl phenyl ether, 5 parts by weight

The above-mentioned amounts of active compound, carrier and emulsifier are crushed and mixed to make a wettable powder. A portion of the wettable powder comprising the prescribed amount of active compound is diluted with water and used for testing.

Testing Procedure

Kidney beans (variety:Serina) were cultivated in plastic pots each having a diameter of 6 cm. The previously prepared solution of the prescribed concentration of active compound was sprayed over the plants in the 1 leaf stage at a rate of 20 ml per pot. One day after spraying, a gel obtained by mixing a suspension of spores of artificially cultured Botrytis cinera and PDA medium was added dropwise to the removed first leaf for infection in a plastic box having a size of 20 while maintaining humidity. 5 days after the inoculation, the infection rate per pot was classified and evaluated according to the following standard, and the control value (%) was calculated. Phytotoxicity was tested at the same time. This test is an average of the results of 3 replications. The evaluation of the infection rate and the calculation method of the control value were conducted as follows:

| Contraction | |
|---|---|
| rate | Significance of disease |
| 0 | No outbreak of disease |
| 0.1 | Elongation of hyphae is observed |
| 1 | Invaded into the leaf to a half of the gel drop |
| 1.5 | Invaded into the leaf more than half but less than the |
| | same area of the gel drop |
| 2 | Invaded into the leaf to about the same area of the gel |
| | drop |
| 3 | Invaded into the leaf broader than the gel drop |

$Control Value ( \% ) = ( 1 - \frac{Infection rate of treated section}{Infection rate of untreated section} ) \times 100$

Test Results

Compounds No. I-6, I-11, I-18, I-20, I-73, I-75, I-76, I-94, I-95, I-104, I-110, I-112, I-142, I-146 and I-175 showed control values of more than 80% at an active compound concentration of 500 ppm. No phytotoxicity was observed.

### FORMULATION EXAMPLES

### FORMULATION EXAMPLE I (GRANULES)

25 parts by weight of water were added to a mixture of 10 parts by weight of Compound No. I-76 according to the invention, 30 parts by weight of bentonite (montmorillonite), 58 parts by weight of talc and 2 parts by weight of lignin sulphonic acid salt, and the mixture was kneaded thoroughly. The resulting product was granulated by means of an extrusion granulator to form granules having a size of from 10 to 40 meshes. The granules were dried at a temperature between 40 and 50° C.

### FORMULATION EXAMPLE II (GRANULES)

95 parts by weight of a clay mineral having a particle size distribution within La range of from 0.2 to 2 mm were introduced into a rotary mixer. This product was uniformly wetted by spraying thereto under rotation a mixture of 5 parts by weight of Compound No. I-95 according to the invention and a liquid diluent. The granules obtained in this manner were dried at a temperature between 40 and 50° C.

### FORMULATION EXAMPLE III (EMULSIFIABLE CONCENTRATE)

An emulsifiable concentrate was prepared by mixing 30 parts by weight of Compound No. I-175 according to the invention, 51 parts by weight of xylene, 8 parts by weight of polyoxyethylene alkyl phenyl ether and 7 parts by weight of calcium alkylbenzene sulphonate with stirring.

### FORMULATION EXAMPLE IV (WETTABLE POWDER)

A wettable powder was prepared by thoroughly mixing 15 parts by weight of Compound No. I-110according to the invention, 80 parts by weight of a mixture (1:5) of White Carbon (fine powder of hydrated non-crystalline silicon oxide) and powdery clay, 2 parts by weight of sodium alkylbenzene sulphonate and 3 parts by weight of a condensate of sodium alkylnaphthatene sulphonate and formaldehyde in powdery state.

### FORMULATION EXAMPLE V (WATER DISPERSIBLE GRANULES)

20 parts by weight of Compound No. I-112 according to the invention; 30 parts by weight of sodium lignin sulphonate, 15 parts by weight of bentonite and 35 parts by weight of calcined diatomaceous earth powder were thoroughly mixed with water. The resulting product was granulated by means of extrusion through a 0.3 mm screen. After drying the product, water dispersible granules were obtained.

1. Triazolopyrimidines of the formula 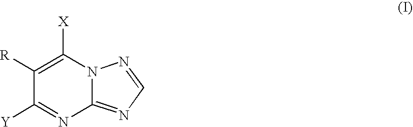
wherein
X represents halogen,
Y represents a hydrogen atom or halogen, and
R represents phenyl-C₁₋₄ allyl optionally substituted by one or more radicals selected from halogen, alkyl, alkenyl, alkylene, dialkylamino, alkoxy, alkylcarbonyl having 1 to 4 carbon atoms in the alkyl group, alkoxycarbonyl having 1 to 4 carbon atoms in the alkoxy group, alkythio, haloalkyl, haloalkoxy, haloalkylthio, phenyl, benzyl, phenoxy, cyano, nitro and thiadiazolyl,
or
R represents diphenylmethyl optionally substituted by one or more radicals selected from halogen, alkyl, alkenyl, alkylene, dialkylamino, alkoxy, alkylcarbonyl having 1 to 4 carbon atoms in the alkyl group alkoxycarbonyl having 1 to 4 carbon atoms in the alkoxy group, alkylthio, haloalkyl, haloalkoxy, haloalkylthio, phenyl, benzyl, phenoxy, cyano, nitro and thiadiazolyl,
or
R represents naphthylmethyl optionally substituted by one or more radicals selected from halogen, alkyl, alkenyl, alkylene, dialkylamino, alkoxy, alkylcarbonyl having 1 to 4 carbon atoms in the alkyl group, alkoxycarbonyl having 1 to 4 carbon atoms in the alkoxy group, alkylthio, haloalkyl, haloalkoxy, haloalkylthio, phenyl, benzyl, phenoxy, cyano, nitro and thiadiazolyl, or
R represents anthranyl-methyl.

2. Triazolopyrimidines of the formula (I) according to claim 1, in which
X represents chloro or bromo,
Y represents a hydrogen atom, chloro or bromo and
R represents phenyl-C₁₋₄ alkyl, optionally substituted by 1 to 5 identical or different radicals selected from fluoro, chloro, bromo, iodo C₁₋₄ alkyl, vinyl, dimethylamino, methoxy, methylcarbonyl, methoxycarbonyl, ethoxycarbonyl, methylthio, difluoromethyl, trifluoromethyl, C₁₋₂ fluoroalkoxy having 1 to 3 fluorine atoms, C₁₋₂ fluoroalkylthio having 1 to 3 fluorine atoms, phenyl, benzyl, phenoxy, cyano, nitro and 1,2,3-thiadiazol-4-yl, or substituted by 1 radical selected from trimethylene and tetramethylene,
R represents diphenylmethyl, each of the phenyl groups being optionally substituted by 1 to 3 identical or different radicals selected from fluoro, chloro, bromo, iodo, C₁₋₄ alkyl vinyl, dimethylamino, methylcarbonyl, methoxycarbonyl, ethoxycarbonyl, methoxy, methylthio, difluoromethyl, trifluoromethyl, C₁₋₂ fluoroalkoxy having 1 to 3 fluorine atoms, C₁₋₂ fluoroalkylthio having 1 to 3 fluorine atoms, phenyl, benzyl, phenoxy, cyano, nitro and 1,2,3-thiadiazol-4-yl, or being substituted by 1 radical selected from trimethylene and tetramethylene,
R represents naphthylmethyl, optionally substituted by 1 or 2 identical or different radicals selected from fluoro, chloro, bromo, iodo, C₁₋₄ alkyl, vinyl, dimethylamino, methylcarbonyl, methoxycarbonyl, ethoxycarbonyl, methoxy, methylthio, difluoromethyl, trifluoromethyl, C₁₋₂ fluoroalkoxyx having 1 to 3 fluorine atoms, C₁₋₂ fluoroalkylthio having 1 to 3 fluorine atoms, cyano and nitro,
or
R represents anthranylmethyl.

3. Triazolopyrimidines of the formula (I) according to claim 1, in which
X represents chloro or bromo,
Y represents a hydrogen atom, chloro or bromo and
R represents phenyl-C₁₋₄ alkyl, optionally substituted by 1 to 5 identical or different radicals selected from fluoro, chloro, bromo, iodo, C₁₋₄ alkyl, vinyl, dimethylamino, methoxy, methylcarbonyl, methoxycarbonyl, ethoxycarbonyl, methylthio, difluoromethyl, trifluoromethyl, trifluoromethoxy, 2,2,2-trifluoroethoxy, trifluoromethylthio, 2,2,2-trifluoroethylthio, phenyl, benzyl, phenoxy, cyano, nitro and 1,2,3-thiadiazol4-yl, or substituted by 1 radical selected from trimethylene and tetramethylene,
or
R represents diphenylmethyl, each of the phenyl groups being optionally substituted by 1 to 3 identical or different radicals selected from fluoro, chloro, bromo, C₁₋₄ alkyl, vinyl, dimethylamino, methoxy, methylcarbonyl, methoxycarbonyl, methylthio, difluoromethyl, trifluoromethyl, trifluoromethoxy, 2,2,2-trifluoroethoxy, trifluoromethylthio, 2,2,27-trifluoroethylthio, phenyl, benzyl, phenoxy, cyano, nitro and 1,2,3-thiadiazol-4-yl,
or
R represents naphthylmethyl, optionally substituted by 1 or 2 identical or different radicals selected from fluoro, chloro, bromo, C₁₋₄ alkyl, dimethylamino, methylcarbonyl, methoxycarbonyl, methoxy, methylthio, difluoromethyl, trifluoromethyl, trifluoromethoxy, trifluoromethylthio, cyano and nitro,
or
R represents anthranylmethyl.

4. Triazolopyrimidines of the formula (I) according to claim 1, in which
X is chloro or bromo,
Y is chloro or bromo and
R represents substituted phenyl C₁₋₄ alkyl optionally substituted diphenylmethyl or optionally substituted naphthylmethyl.

5. Triazolopyrimidines of the formula (I) according to claim 1, in which
X represents chloro,
Y represents a hydrogen atom or chloro and
R represents optionally substituted phenyl C₁₋₄ alkyl.

6. Triazolopyrimidines of the formula (I) according to claim 1, in which
X represents chloro,
Y represents chloro and
R represents phenyl C₁₋₄ alkyl, which is substituted by 1 to 5 identical or different radicals selected from the phenyl radicals mentioned in claim 3.

7. Process for the preparation of triazolopyrimidines of the formula (I) according to claim 1, characterized in that
a) in a first step compounds of the formula: 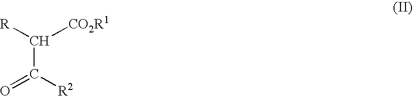
wherein
R has the above-mentioned meanings,
R¹ represents C₁₋₄ alkyl, and
R² represents a hydrogen atom or C₁₋₄ alkoxy,
are reacted with 3 amino-1,2,4-triazole of the formula 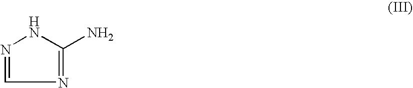
in the presence of an inert diluent and, if appropriate, in the presence of an acid-binding agent or of an acid catalyst,
and
b) reacting in a second step the triazolopyrimidines thus obtained having the formula 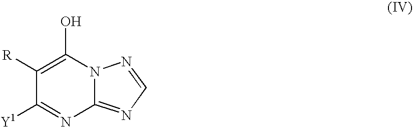
wherein
R has the above-mentioned meanings and
Y¹ is a hydrogen atom or hydroxy,
are reacted with halogenating agents in the presence of a diluent.

8. Microbicidal compositions, characterized in that they contain at least one triazolopyrimidine of the formula (I) according to claim 1 plus extenders and/or surface-active agents.

9. Process for combating undesired microorganisms, characterized in that triazolopyrimidines of the formula (I) according to claim 1 are applied to the microorganisms and/or to their habitat.

10. Use of triazolopyrimidines of the formula (I) according to claim 1 for combating undesired microorganisms.

11. Process for the preparation of microbicidal compositions, characterized in that triazolopyrimidines of the formula (I) according to claim 1 are mixed with extenders and/or surface-active agents.
